(19) United States
(12) Patent Application Publication
(10) Pub. No.: US 2005/0154306 A1
(43) Pub. Date: Jul. 14, 2005

(54) Dort process-based method and system for adaptive beamforming in estimating the aberration in a medium

(76) Inventors: Michael Burcher, Tarrytown, NY (US); Anna T. Fernandez, Croton-On-Hudson, NY (US); Claude Cohen-Bacrie, New York, NY (US)

Correspondence Address: PHILIPS INTELLECTUAL PROPERTY & STANDARDS, P.O. BOX 3001, BRIARCLIFF MANOR, NY, 10510, US

(21) Appl. No.: 11/034,663

(22) Filed: Jan. 13, 2005

Related U.S. Application Data
(60) Provisional application No. 60/536,690, filed on Jan. 14, 2004.
(60) Provisional application No. 60/614,720, filed on Sep. 30, 2004.

(51) Int. Cl. A61B 8/02

(52) U.S. Cl. 600/447

(57) ABSTRACT

A method and system for adapting a focused beam of focused ultrasound imaging signals from an ultrasound probe (14) for compensating for the presence of an aberration (X) in a medium (OBJ) transmits a focused ultrasound imaging signal (FOC) into a region of a medium using a plurality of focused beams from a plurality of probe array elements (P). The return focused ultrasound imaging signals are received and a focused decomposition of time-reversal operator process determines the presence of an aberration in the region and correlates the aberration to selected probe array elements. The method and system adapt selected characteristics associated with the transmission of the ultrasound imaging signals and reception of the return focused ultrasound imaging signals from the selected probe array elements for compensating for the presence of the aberration in the region. The method and system further involve transmitting adapted focused ultrasound imaging signals into the region for generating a homogeneous wave front having reduced inhomogeneities arising from the aberration.

### BACKGROUND OF THE INVENTION

The invention relates to a method for analyzing an organic medium potentially including defects within a noisy structure, the medium being excited by an ultrasonic signal emitted by a set of transducers. More particularly, the present invention relates a DORT process-based method and system for adaptive beamforming that estimates an aberration in a medium, as may be particularly applied to medical and ultrasonic imaging systems.

In medical ultrasound imaging, focusing beams allows their transmission and receipt along multiple distinct lines to form an image. This focusing assumes a uniform sound speed within the medium. In reality, however, ultrasonic waves propagate through different tissue types with different speeds. These variations in sound speed aberrate the wave fronts and prevent them from properly focusing. The image resulting from these aberrated wave fronts is degraded, exhibiting poor spatial and contrast resolution.

A number of methods have been proposed to correct the effects of aberration, and thus improve image quality. In several of these, the effect of the aberrator is modeled as a near-field phase screen which acts to delay the wave front differently across the aperture. If the phase-screen is known, then the aberration can be corrected by appropriately adjusting the focusing delays, thereby realigning the wave fronts. The adjustment could feasibly be accomplished in a modem ultrasound machine and some early implementations show improved image quality. In such a process, the phase-screen aberrator must first be estimated in order to calculate the appropriate correcting delays. This is typically done in a two-stage process.

In the first stage, the signals at adjacent or near-adjacent pairs of elements are correlated in order to find the differential time lag which best aligns them. In the second stage, these measurements of differential time lag are combined to estimate the aberrator which best matches the measurements using a least squares method. The resulting aberration estimate has a tendency to track the incident wave fronts. When the measurement is carried out in a speckle region or on a point target, the wave front will resemble the aberration profile. However, when there are multiple close scatterers, their wave fronts will interfere with one another and a wave front-following process will not yield an accurate estimate of the aberrator.

The article, “Ultrasonic Nondestructive Testing of Scattering Media using the Decomposition of the Time-Reversal Operator,” published in IEEE Transactions on Ultrasonics, Ferroelectrics and Frequency Control, Vol. 49, No 8, August 2002, by E. Kerbrat, C. Prada, D. Cassereau & M. Fink, (hereinafter “Kerbrat”) references a way to study a medium using the DORT method (which is a French acronym for “diagonalization of the time reversal operator”). In that process, a first transducer in an array of transducers is excited by a short excitation and the signals resulting from the response of the medium are received on all the transducers in the array. This operation is repeated for each of the transducers with the same excitation. A square transfer matrix K is then obtained by producing a Fourier transform of the responses of the medium. The time-reversal operator is then defined by K*K, which is the conjugate transpose of the matrix, and can be diagonalized. The number of significantly non-zero proper values is equal to the number of defects detected by the method, which defects are then located using a calculation of the proper vectors.

For the case of two well-resolved, point-like scatterers, the process of Kerbrat identifies the focal laws required to focus on each of them, even though their wave fronts overlapped. This process, like other known methods for the detection of small-size defects in medical ultrasonic imaging have the drawback of requiring many excitations particular to the method. The particular character of these excitations makes it possible to take account of only some of the information present in the medium. Such methods must therefore be used in “parallel with and independently of” other insonifications of the medium to enable having access to other information in order to obtain, for example, an image of the medium. In addition, these particular excitations cannot be carried out by a common ultrasonic imaging apparatus and the use of a specific apparatus is therefore obligatory.

### SUMMARY OF THE INVENTION

The present invention provides for adaptive beamforming based on the DORT method for estimating the aberration in a medium. As such, the present invention overcomes or substantially eliminates the problems associated with prior methods estimating an aberration in a medium, as may be particularly applied to medical and ultrasonic imaging systems.

The DORT method provides a detection technique based on the time-reversal operator which has been widely used in non-destructive testing. The present invention adapts the DORT method to an imaging mode, wherein transmissions in the medium are focused waves and a windowing preprocessing operation on the received signals significantly increases the sensitivity. Alternatively, different aberration profiles can be measured at different points in the image and used to correct the beamforming delay table for that part, or adjacent parts of the image.

A technical advantage of the present invention is the ability to perform phase aberration correction. If there were no aberration present, then the delays would be determined by the geometry of the transducer and would form a parabola. This simple parabolic delay law is currently used in nonadaptive imaging to focus the received signals. If an aberration is present, then the focal law required to focus on the target will not be parabolic. The difference between the delay law found from the K matrix and the parabolic delay law is therefore an estimate of the aberration profile. The aberration estimate can be used to correct the global image beamforming delay table.

Another technical advantage of the present invention is the ability to achieve a speed of sound correction in which the focalization laws for several structures in the medium are processed to estimate the global speed of sound in the medium. The new global speed of sound could then be used to recalculate the delay tables used in beamforming and produce better-focused images of the medium.

Still another technical advantage of the present invention is the ability to provide image stabilization. For surgical procedures the algorithm could be initialized on a cell centered on the catheter. Once the focalization law corresponding to the catheter had been identified, the region could be imaged using focal laws centered on it. The aim of this would be to generate a clearer, better-focused image of the catheter.

Other objects, features and advantages of the invention will be apparent from the following specification taken in conjunction with the following drawings.

For a more complete understanding of the present DORT process-based method and system for adaptive beamforming In estimating the aberration in a medium, reference is now made to the following description which is to be taken in conjunction with the accompanying drawings and in which like reference numbers indicate like features and further wherein:

### BRIEF DESCRIPTION OF THE DRAWINGS

FIG. 1 is an illustration of an ultrasound device which may be used to implement the methodologies disclosed herein;

FIG. 2 is a schematic diagram illustrating the functional elements of a device of the type depicted in FIG. 1;

FIG. 3 is a diagram explaining the reception according to the invention of the echo graphic signals coming from a medium and the formation of the rectangular response matrix;

FIG. 4 is a partial functional diagram of an ultrasonic imaging apparatus implementing the invention,

FIG. 5 is a graph of the singular values obtained according to the first embodiment of the invention,

FIG. 6 illustrates the location of a singular zone according to the invention,

FIG. 7 illustrates an application of the invention to an ultrasonic image,

FIG. 8 is a general functional diagram of an apparatus for analyzing a medium according to the invention; and

FIG. 9 provides a process flow diagram for the process of the present invention.

### DESCRIPTION OF THE PREFFER EMBODIMENTS

The description which follows is presented so as to enable a person skilled in the art to implement and make use of the invention. Various alternatives to the preferred embodiment will be obvious to a person skilled in the art and the generic principles of the invention disclosed here may be applied to other embodiments. Thus the present invention is not deemed to be limited to the embodiment described, but rather to have the widest scope in accordance with the principles and characteristics described below.

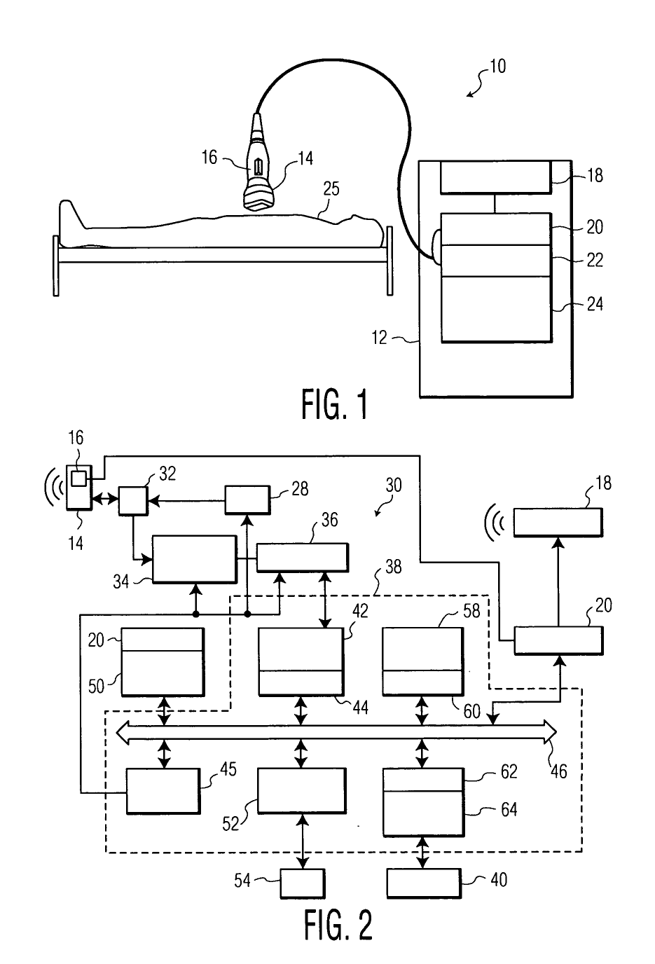

FIG. 1 shows a simplified block diagram of an ultrasound imaging system 10 that may be used in the implementation of the methodologies disclosed herein. It will be appreciated by those of ordinary skill in the relevant arts that ultrasound imaging system 10, as illustrated in FIG. 1, and the operation thereof as described hereinafter, is intended to be generally representative of such systems and that any particular system may differ significantly from that shown in FIG. 1, particularly in the details of construction and in the operation of such system. As such, ultrasound imaging system 10 is to be regarded as illustrative and exemplary, and not limiting, as regards the methodologies and devices described herein or the claims attached hereto.

Ultrasound imaging system 10 generally includes ultrasound unit 12 and connected transducer 14. Transducer 14 includes spatial locator receiver (or simply “receiver”) 16. Ultrasound unit 12 has integrated therein spatial locator transmitter (or simply “transmitter”) 18 and associated controller 20. Controller 20 provides overall control of the system by providing timing and control functions. As will be discussed in detail below, the control routines include a variety of routines that modify the operation of receiver 16 so as to produce a volumetric ultrasound image as a live real-time image, a previously recorded image, or a paused or frozen image for viewing and analysis.

Ultrasound unit 12 is also provided with imaging unit 22 for controlling the transmission and receipt of ultrasound, and image processing unit 24 for producing a display on a monitor (See FIG. 2). Image processing unit 24 contains routines for rendering a three-dimensional image. Transmitter 18 is preferably located in an upper portion of ultrasound unit 12 so as to obtain a clear transmission to receiver 16. Although not specifically illustrated, the ultrasound unit described herein may be configured in a cart format.

During freehand imaging, a user moves transducer 14 over subject 25 in a controlled motion. Ultrasound unit 12 combines image data produced by imaging unit 22 with location data produced by the controller 20 to produce a matrix of data suitable for rendering onto a monitor (See FIG. 2). Ultrasound imaging system 10 integrates image rendering processes with image processing functions using general purpose processors and PC-like architectures. On the other hand, use of ASICs to perform the stitching and rendering is possible.

FIG. 2 is a block diagram 30 of an ultrasound system that may be used in the practice of the methodologies disclosed herein. The ultrasound imaging system shown in FIG. 2 is configured for the use of pulse generator circuits, but could be equally configured for arbitrary waveform operation. Ultrasound imaging system 10 uses a centralized architecture suitable for the incorporation of standard personal computer (“PC”) type components and includes transducer 14 which, in a known manner, scans an ultrasound beam, based on a signal from a transmitter 28, through an angle. Backscattered signals, i.e., echoes, are sensed by transducer 14 and fed, through receive/transmit switch 32, to signal conditioner 34 and, in turn, to beamformer 36. Transducer 14 includes elements which are preferably configured as a steerable two-dimensional array. Signal conditioner 34 receives backscattered ultrasound signals and conditions those signals by amplification and forming circuitry prior to their being fed to beamformer 36. Within beamformer 36, ultrasound signals are converted to digital values and are configured into “lines” of digital data values in accordance with amplitudes of the backscattered signals from points along an azimuth of the ultrasound beam.

Beamformer 36 feeds digital values to application specific integrated circuit (ASIC) 38 which incorporates the principal processing modules required to convert digital values into a form more conducive to video display that feeds to monitor 40. Front end data controller 42 receives lines of digital data values from beamformer 36 and buffers each line, as received, in an area of buffer 44. After accumulating a line of digital data values, front end data controller 42 dispatches an interrupt signal, via bus 46, to shared central processing unit (CPU) 48, CPU 48 executes control procedures 50 including procedures that are operative to enable individual, asynchronous operation of each of the processing modules within ASIC 38. More particularly, upon receiving an interrupt signal, CPU 48 feeds a line of digital data values residing in buffer 42 to random access memory (RAM) controller 52 for storage in random access memory (RAM) 54 which constitutes a unified, shared memory. RAM 54 also stores instructions and data for CPU 48 including lines of digital data values and data being transferred between individual modules in ASIC 38, all under control of RAM controller 52.

Transducer 14, as mentioned above, incorporates receiver 16 that operates in connection with transmitter 28 to generate location information. The location information is supplied to (or created by) controller 20 which outputs location data in a known manner. Location data is stored (under the control of the CPU 48) in RAM 54 in conjunction with the storage of the digital data value.

Control procedures 50 control front end timing controller 45 to output timing signals to transmitter 28, signal conditioner 34, beamformer 36, and controller 20 so as to synchronize their operations with the operations of modules within ASIC 38. Front end timing controller 45 further issues timing signals which control the operation of the bus 46 and various other functions within the ASIC 38.

As previously noted, control procedures 50 configure CPU 48 to enable front end data controller 44 to move the lines of digital data values and location information into RAM controller 52, where they are then stored in RAM 54. Since CPU 48 controls the transfer of lines of digital data values, it senses when an entire image frame has been stored in RAM 54. At this point, CPU 48 is configured by control procedures 50 and recognizes that data is available for operation by scan converter 58. At this point, therefore, CPU 48 notifies scan converter 58 that it can access the frame of data from RAM 54 for processing.

To access the data in RAM 54 (via RAM controller 52), scan converter 58 interrupts CPU 48 to request a line of the data frame from RAM 54. Such data is then transferred to buffer 60 of scan converter 58 and is transformed into data that is based on an X-Y coordinate system. When this data is coupled with the location data from controller 20, a matrix of data in an X-Y-Z coordinate system results. A four-dimensional matrix may be used for 4-D (X-Y-Z-time) data. This process is repeated for subsequent digital data values of the image frame from RAM 54. The resulting processed data is returned, via RAM controller 52, into RAM 54 as display data. The display data is typically stored separately from the data produced by beamformer 36. CPU 48 and control procedures 50, via the interrupt procedure described above, sense the completion of the operation of scan converter 58. Video processor 62, such as the MITSUBISHI VOLUMEPRO series of cards, interrupts CPU 48 which responds by feeding lines of video data from RAM 54 into buffer 62, which is associated with the video processor 64. Video processor 64 uses video data to render a three-dimensional volumetric ultrasound image as a two-dimensional image on monitor 40.

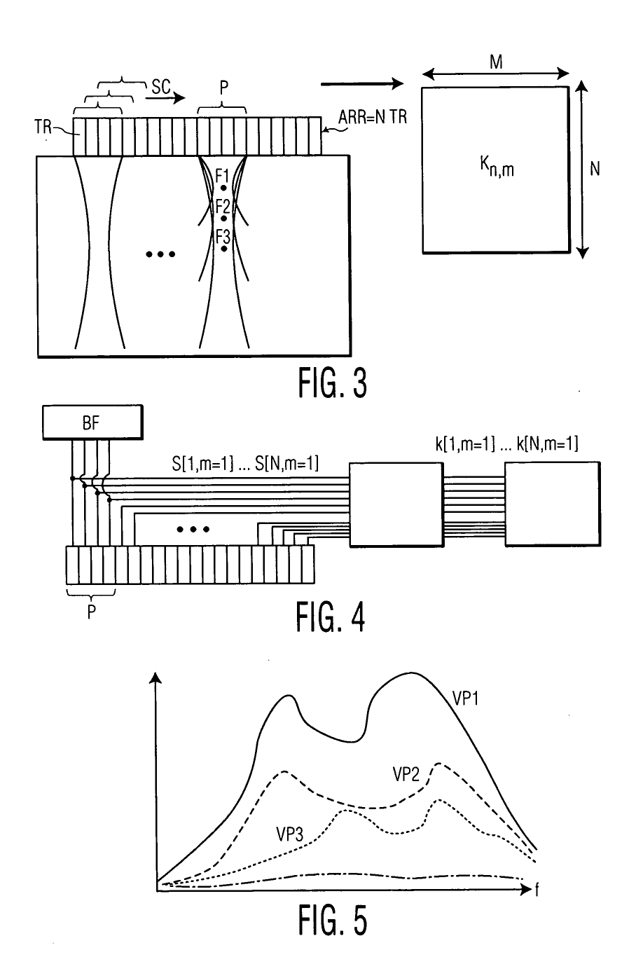

FIG. 3 depicts a diagram explaining the reception of echographic ultrasonic signals coming from a medium MID according to the requirements of the invention. The medium MID is excited by focused ultrasonic waves FOC. The focusing is carried out on P transducers TR in an array of transducers ARR centered on the geometric middle of these P transducers. According to FIG. 3, P=4. The focusing techniques also make it possible to center the wave at any point on the array of transducers. According to the invention, the echographic signals returned by the medium MID are then recorded on each of the N transducers AR in the array ARR of transducers TR. According to the ultrasonic acquisition methods conventionally used in medical imaging, the excitation is then repeated according to the same focusing on P transducers but offset with respect to the previous one in a scanning direction indicated for example in FIG. 3 by the arrow SC. Thus, according to a conventional scanning, M acquisitions are made.

The acquisition number M may vary and is generally different from the number N of transducers TR in the array AR of transducers TR. In addition, the conventional acquisitions are made generally and according to an advantageous embodiment of the invention for various focusing depths represented by the points F1, F2, and F3. Each acquisition at a given depth gives particular information at said depth. However, these excitations do not make it possible to access the inter-element responses to a pulse which are essential to the implementation of the proposed method of the prior art.

FIG. 4 is a partial functional diagram of an ultrasonic imaging apparatus implementing the invention. This functional diagram shows more particularly an acquisition made according to the invention using a first excitation m=I made by a broad frequency spectrum wave focused on the first P transducers in the array of transducers. For example, the spectrum is centered on a frequency of 3 to 5 MHz and possesses a bandwidth of 40% of the total bandwidth. The echo graphic signals S(n=1,m=1]. . . S(n=N,m=1] are received independently by each of the transducers n, n ε[1,N] following the excitation m=1 and are transmitted to beam formation means BF so as then to generate an ultrasonic image according to the means known to persons skilled in the art. This ultrasonic image generally makes it possible, referring to FIG. 7, to see the medium MID and an object OBJ included in this medium MID but does not in general make it possible to distinguish a defect X of small size in the generally noisy image (notably presenting a speckle noise). This object may for example be an organ and the small defect may be a reflector due to the presence of an ailing area. Thus, with the breast, the small defect may be a microcalcification. According to the invention, the echographic signals S [1,m=1]. . . S[N,m=1] (also denoted Sn1) received independently by each of the transducers n in the entire array of transducers are also transmitted to a selector SEL which selects a time part of the signal received at each transducer.

This time part generally corresponds to the signals received coming from the vicinity of a focusing point F(l, 2 or 3) as presented in FIG. 3. But could be from a region at a significantly different depth from the focusing point. The magnitude of this vicinity depends on the compromise which the user wishes to have between detection at a great depth and the precision of this detection. A scanning of m excitations is then carried out along the array of transducers according to the techniques conventionally used in medical ultrasonic imaging. Time parts of the signals received S [1,m] . . . S [N,m] are selected for each of the excitations m of a scanning of the medium of M excitations, m thus being included in [I,M]. These signal time parts are denoted knm or k[n,m] and are time functions. These signal parts k[n,m] are then transmitted to a module PEM for the particular exploitation of the signals. For each excitation m of the scanning, this module PEM stores in memory the part knm of the response function Snm received by a transducer n from the medium MID corresponding to a certain depth interval. The Fourier transforms of the signals kₙₘ(t) give the matrix K=(Kₙₘ(ro)) which is referred to as the response matrix. Thus a matrix of coefficients Knm is obtained each representing the response of the medium for a given excitation frequency received by the element n following a focused excitation m of the medium. This matrix is rectangular and can be calculated for each frequency of the spectrum, generally according to a making discrete thereof.

It is possible to acquire only one matrix for a single frequency but the result may be less precise. The frequencies chosen may also be selected in order to comply with the constraints of resolution and attenuation in the medium. Such frequencies are in fact dictated, according to the invention, by the acquisition of the conventional ultrasonic image of the medium. Although it may be possible to use different parameters for the DORT acquisition, such as a different bandwidth or center frequency, compared with the imaging acquisition. The DORT transmissions would still use focused transmissions of the sort that normally used for imaging. However, it is possible to form the image with a different set of transmissions. In fact, the transmissions may be interleaved with one another.

The module PEM next calculates the decomposition into singular values of the response matrix K. Effectively, this decomposition is in particular used for the resolution of a singular system and a rectangular matrix of dimension NM with real or complex coefficients may be decomposed in the form K=UDV with U the unitary matrix of dimension NN and V the unitary matrix of dimension MM and D a diagonal matrix of dimension NM. The diagonal elements of the matrix D of dimension NM are simply the square roots of the singular values of the matrix K*K where K* is the conjugate of the transpose.

The singular vectors of the matrix K*K are the columns of V. The singular vectors of the matrix KK* are the columns of U. In a first embodiment, the module PEM calculates a plurality of response matrices for a plurality of frequencies. In this case a graph representing the amplitude AMP of the singular values VP according to the frequency f as presented in FIG. 5 is obtained. The singular values VP1, VP2, VP3 do not all appear with the same relative intensities for the same frequency values. A defect in a medium is marked by a local change in reflection of the signal and therefore can be considered and defined by the reflector term.

The correspondence between the presence of a reflector and the presence of a non-zero singular value was studied, for the simple method of diagonalization of the time-reversal operator, in the document “Eigenmodes of the Time-Reversal Operator: A Solution to Selective Focusing in Multiple Target Media”, C. Prada, M. Fink, Wave Motion 20/1994, pp. 151-183. It is observed that the invention makes it possible also to obtain this correspondence. Thus, according to the invention, a non-zero singular value of the rectangular matrix constructed in the frequency domain studied reveals the presence of a reflector. The correspondence is therefore a singular value=a reflector and the largest singular value corresponds to the largest reflector.

According to the first embodiment, a plurality of matrices is constructed for various frequencies and an inverse Fourier transform of the proper vectors, that is to say the columns of the matrix D, can then be calculated. This makes it possible to obtain the singular time vectors which correspond to the singular frequency vectors. This makes it possible to propagate simply in return the singular time vectors in the medium so as to determine the pressure fields whose maxima correspond to the defects in the medium. This return propagation of the singular vectors in phase and amplitude uses for example the reception focusing techniques and is generally done by digital means which simulate the acoustic field within the medium. In practice software performs this function of notional time wave propagation.

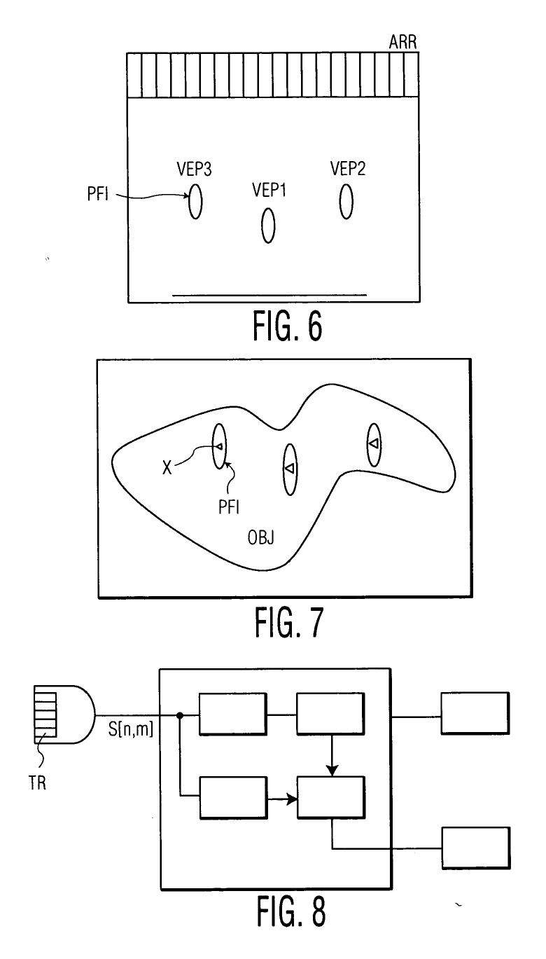

For example, in FIG. 6, this first technique of propagation of a singular time vector results in coloring in a certain way the high pressure zones PFI. An image with color levels can also be obtained. Software can also be proposed for reconstructing a propagation matrix for each depth of the medium thus made discrete. A matrix for passage from the plane of the sensor to the plane of a given depth is then obtained, making it possible to locate a reflector on the dimension Y. This reconstruction for various depths can, using the advantageous embodiment, be the result of an insonification of the medium according to waves focused at various depths and in a way which is made discrete. For example, in FIG. 3, three focusings of depth F1, F2, F3 are effected and a propagation matrix is constructed for these three depths.

The two techniques of locating the singular zones proposed above make it possible to isolate one zone and in practice to display a pressure field PFI on a conventional ultrasonic image of a medium MID including an object OBJ as presented in FIG. 7. A marking is therefore carried out on an ultrasonic image of the zone studied in order to locate the defect or defects. For example, by virtue of the invention, a micro calcification is detectable in the breast whereas it may not emerge from the noise (“speckle”) on a conventional ultrasonic image.

The invention can be implemented for all types of medical imaging with ultrasonic acquisition. It is possible to use, according to the invention, the responses given by an organic medium after excitations according to various types of focusing used in ultrasonic imaging. The line density (that is to say the geometric interval between two successive excitations) may also be adapted independently of the invention so as to make the results given by the invention more precise laterally. It is thus possible to have a number M greater than N. In this case the system to be resolved is degenerated.

The invention can also be used to form an adaptable insonification beam: the singular vectors make it possible to send a strong pressure field on a tricky area and” consequently make it possible to have more precise information on this area.

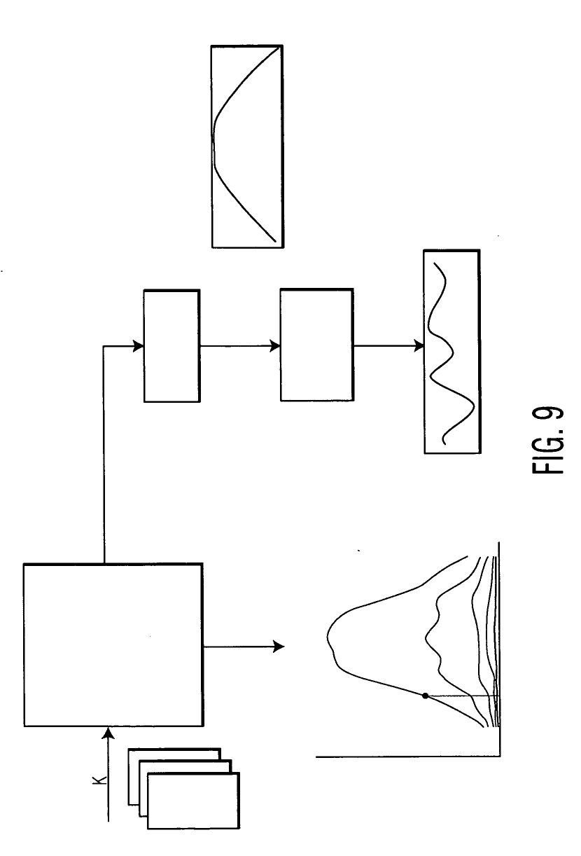

FIG. 8 depicts schematically an apparatus in which a method according to the invention is implemented. The invention can be implemented non-removably or in a modular Form, a reflector detection module being added to a conventional ultrasonic apparatus. This detection module receives the echographic signals independently of the transducers of the ultrasonic apparatus and includes, for example, a selector SEL and an exploitation module PEM as described above. In FIG. 8, an apparatus in which the invention is permanently is depicted.

This apparatus includes a probe PROB including reception elements TR, said probe being connected by conventional means to a data processing apparatus LAB. In addition to a module BF for forming a return beam and an image according to the known techniques of ultrasonic imaging, any data processing apparatus LAB includes a selector SEL and an operating module PEM as described previously. The apparatus LAB is connected to a display module DIS which displays, by means of conventional display functions, in addition to the images conventionally obtained by an ultrasonic apparatus, the images which can be constructed from information obtained by virtue of the module PEM. A combination module CMB combines for example the data obtained by beam formation means BF and those obtained by the operating module PEM. Next the combination module CMB is connected to the display module DIS.

Thus an image as presented in FIG. 7 and locating the singular zones can be obtained according to the invention. Any means of graphical representation of the singular zones (binary image, surrounding the zone etc) can be used indifferently for application of the invention. A user interface VIF is advantageously connected to the apparatus LAB for controlling this apparatus and parameterizing it: for example, a detection threshold value can be modified by the user as well as the depth incrementation value which can determine the precision of the location/detection of a reflector, the object of the invention.

The present invention provides a further development on the DORT process. For every transmitted beam that crosses the cell, the received signals from each element are windowed in time so that they only contain information from the depths corresponding to the cell. (The other transmitted beams and regions outside the window are ignored.) These windowed signals are then Fourier transformed. For each frequency, fo, in a given set, a matrix K(fo)is computed with a number of columns equal to the number of transmitted beams crossing the cell, and a number of lines equal to the number of individual receiving elements. Each element of the matrix is the (complex) coefficient of the Fourier transform of the signal from the corresponding transmitting beam and receiving element, at the given frequency.

The present invention further provides a method for adapting a focused beam of focused ultrasound imaging signals from an ultrasound probe for compensating for the presence of an aberration in a medium. This is accomplished by transmitting focused ultrasound imaging signals into a region of a medium using a plurality of focused beams from a plurality of probe array elements and receiving return focused ultrasound imaging signals from said region. The process further includes applying the focused DORT process for determining the presence of an aberration in said region and correlating said aberration to selected probe array elements in said plurality of probe array elements. Then, the process adapts selected characteristics associated with the transmission of said focused ultrasound imaging signals and reception of said return focused ultrasound imaging signals from said selected probe array elements for compensating for the presence of said aberration in said region. This further permits transmitting adapted focused ultrasound imaging signals into said region for generating a homogeneous wave front having reduced inhomogeneities arising from said aberration.

The singular value decomposition of the matrix K(fo), therefore, gives a diagonal matrix containing the eigenvalues of K and a matrix containing the eigenvectors of K in the receive basis. The eigenvalues of K indicate the reflectivity of point targets within the cell. The phase of the complex eigenvectors can be translated into a delay which varies over the receive elements. These delays correspond to the focal laws which would focus on the point targets.

This focused DORT method accommodates breast microcalcification detection and array adaptation. While the present invention applies to curved, phased, and linear arrays, the process may be used on a linear array by the following process. The process begins by first acquiring K(fo). To measure the aberration at the lateral position P, N parallel focused beams are transmitted into the medium, as shown in FIG. 9, The beams are evenly spaced and centered about P. For each transmission, the signals on all L receive elements are recorded. The signals are gated in time, keeping only the signals from depths z−□z to z+□z. For the nth transmission beam, this is achieved using the geometric focal law which focuses along beam n at depth z. A temporal Fast Fourier Transform is performed on each of the L×N signals, resulting in a matrix K(f) at several frequencies.

Then, DORT is performed on the acquired K(f) matrix. This gives a set of singular values and their corresponding eigenvectors. At each frequency, a focal law is measured by unwrapping the phase of the first eigenvector and multiplying it by the period, 1/f.

Average aberration estimates are then taken over frequency. The measured focal laws are averaged over the frequencies within the bandwidth of the transducer. The measured aberration profile is then the difference between the frequency-averaged focal law and the geometric focal law. Alternatively, different aberration profiles can be measured at different points in the image and used to correct the beamforming delay table for that part, or adjacent parts of the image.

The invention makes it possible to obtain precise location of any reflecting defects within a homogeneous medium for which noisy signals (“speckle”) are obtained, signals within which it is generally difficult to detect such defects with the known mean. In one of its applications, the invention advantageously concerns compound imaging consisting of insonifying a medium in different directions and combining the results so as to obtain a more complete and less noisy image.

The modules presented previously for fulfilling the functions presented in the steps of the method according to the invention can be integrated as an additional application in a conventional ultrasonic apparatus or be used in an independent apparatus intended to be connected to a conventional ultrasonic apparatus for fulfilling the functions according to the invention. There exist many ways of implementing the functions presented in the steps of the method according to the invention by software and/or hardware means accessible to persons skilled in the art. Thus, although the Figures show various functions performed by various units, this does not exclude a single software and/or hardware means making it possible to fulfill several functions. Nor does this exclude a combination of software and/or hardware means making it possible to fulfill a function. Although this invention has been described in accordance with the embodiments presented, a person skilled in the art will immediately recognize that there exist variants to the embodiments presented and that these variants remain within the spirit and scope of the present invention. Thus, many modifications can be achieved by a person skilled in the art without for all that being excluded from the spirit and scope defined by the following claims.

1. A method for adapting a focused beam of focused ultrasound imaging signals from an ultrasound probe for compensating for the presence of an aberration in a medium, comprising the steps of:
transmitting focused ultrasound imaging signals into a region of a medium using a plurality of focused beams from a plurality of probe array elements;
receiving return focused ultrasound imaging signals from said region;
applying a focused decomposition of time-reversal operator process for determining the presence of an aberration in said region and correlating said aberration to selected probe array elements in said plurality of probe array elements; and
adapting selected characteristics associated with the transmission of said focused ultrasound imaging signals and reception of said return focused ultrasound imaging signals from said selected probe array elements for compensating for the presence of said aberration in said region.

2. The method of claim 1, further comprising the step of transmitting adapted focused ultrasound imaging signals into said region for generating a homogeneous wave front having reduced inhomogeneities arising from said aberration.

3. The method of claim 1, wherein said adapting step further comprises the step of shifting the phase of selected probe array elements.

4. The method of claim 1, wherein said adapting step further comprises the step of shifting the phase of selected probe array elements on a single element basis.

5. The method of claim 1, further comprising the step of forming a homogeneous wave front signal representing the transmission of said focused ultrasound imaging signals and reception of said return focused ultrasound imaging signals.

6. The method of claim 1, further comprising the step of applying a focused decomposition of time-reversal operator process for determining the presence of an aberration in said region and correlating said aberration to selected probe array elements in said plurality of probe array elements using an eigenvector matrix, said eigenvector matrix providing diagonal data elements relating to the presence of said aberration.

7. The method of claim 6, further comprising the step of adapting the phase of said transmission of said focused ultrasound imaging signals and reception of said return focused ultrasound imaging signals in response to said diagonal data elements.

8. The method of claim 1, further comprising the step of adapting selected characteristics associated with the transmission of said focused ultrasound imaging signals and reception of said return focused ultrasound imaging signals from said selected probe array elements for compensating for the presence of said aberration in said region on a single element basis.

9. The method of claim 8, wherein said ultrasound probe comprises a curved ultrasound probe and further comprising the step of adapting selected characteristics of selected curved probe elements for compensating for the presence of said aberration in said region on a single element basis.

10. The method of claim 8, wherein said ultrasound probe comprises a linear ultrasound probe and further comprising the step of adapting selected characteristics of selected linear probe elements for compensating for the presence of said aberration in said region on a single element basis.

11. The method of claim 8, wherein said ultrasound probe comprises a phased ultrasound probe and further comprising the step of adapting selected characteristics of selected phased probe elements for compensating for the presence of said aberration in said region on a single element basis.

12. The method of claim 1, wherein said medium comprises a human breast and further comprising the step of applying a focused decomposition of time-reversal operator process for determining the presence of a microcalcification in said region of said human breast and correlating said microcalcification to selected probe array elements in said plurality of probe array elements.

13. The method of claim 1, wherein said step of applying a focused decomposition of time-reversal operator process for determining the presence of an aberration in said region and correlating said aberration to selected probe array elements in said plurality of probe array elements further comprises the steps of:
focusing said ultrasound imaging signal at said region at M distinct successive excitations for forming an image of said region after reception of the responses from said medium;
forming a rectangular response matrix of dimension N*M, a coefficient Km of which representing a response of the medium received by the transducer n following an excitation m,
decomposing said response matrix into singular values, and
relating singular vectors to said singular values for locating singular zones corresponding to defects in said medium.

14. An ultrasound imaging system for adapting a focused beam of focused ultrasound imaging signals from an ultrasound probe and compensating for the presence of an aberration in a medium, comprising:
a plurality of focused beams from a plurality of probe array elements for transmitting focused ultrasound imaging signals into a region of a medium;
said probe array elements further for receiving return focused ultrasound imaging signals from said region;
instructions operating in an associated processor for applying a focused decomposition of time-reversal operator process for determining the presence of an aberration in said region and correlating said aberration to selected probe array elements in said plurality of probe array elements; and
circuitry for adapting selected characteristics associated with the transmission of said focused ultrasound imaging signals and the reception of said return focused ultrasound imaging signals from said selected probe array elements for compensating for the presence of said aberration in said region.

15. The system of claim 14, further comprising circuitry for transmitting adapted focused ultrasound imaging signals into said region for generating a homogeneous wave front having reduced inhomogeneities arising from said aberration.

16. The system of claim 14, further comprising circuitry for shifting the phase of selected probe array elements.

17. The system of claim 14, further comprising circuitry for shifting the phase of selected probe array elements on a single element basis.

18. The system of claim 14, further comprising circuitry for forming a homogeneous wave front signal representing the transmission of said focused ultrasound imaging signals and reception of said return focused ultrasound imaging signals.

19. The system of claim 14, further comprising circuitry for applying a focused decomposition of time-reversal operator process for determining the presence of an aberration in said region and correlating said aberration to selected probe array elements in said plurality of probe array elements using an eigenvector matrix, said eigenvector matrix providing diagonal data elements relating to the presence of said aberration.

20. The system of claim 19, further comprising circuitry for adapting the phase of said transmission of said focused ultrasound imaging signals and reception of said return focused ultrasound imaging signals in response to said diagonal data elements.

21. The system of claim 14, further comprising circuitry for adapting selected characteristics associated with the transmission of said focused ultrasound imaging signals and reception of said return focused ultrasound imaging signals from said selected probe array elements for compensating for the presence of said aberration in said region on a single element basis.

22. The system of claim 21, wherein said ultrasound probe comprises a curved ultrasound probe and further comprising further comprising circuitry for adapting selected characteristics of selected curved probe elements for compensating for the presence of said aberration in said region on a single element basis.

23. The system of claim 21, wherein said ultrasound probe comprises a linear ultrasound probe and further comprising further comprising circuitry for adapting selected characteristics of selected linear probe elements for compensating for the presence of said aberration in said region on a single element basis.

24. The system of claim 21, wherein said ultrasound probe comprises a phased ultrasound probe and further comprising further comprising circuitry for adapting selected characteristics of selected phased probe elements for compensating for the presence of said aberration in said region on a single element basis.

25. The system of claim 14, wherein said medium comprises a human breast and further comprising further comprising circuitry for applying a focused decomposition of time-reversal operator process for determining the presence of a microcalcification in said region of said human breast and correlating said microcalcification to selected probe array elements in said plurality of probe array elements.

26. The method of claim 13, further comprising circuitry for applying a focused decomposition of time-reversal operator process for determining the presence of an aberration in said region and correlating said aberration to selected probe array elements in said plurality of probe array elements, said circuitry further comprising:
circuitry for focusing said ultrasound imaging signal at said region at M distinct successive excitations for forming an image of said region after reception of the responses from said medium;
circuitry for forming a rectangular response matrix of dimension N*M, a coefficient Kₙₘ of which representing a response of the medium received by the transducer n following an excitation m,
circuitry for decomposing said response matrix into singular values, and
circuitry for relating singular vectors to said singular values for locating singular zones corresponding to defects in said medium.
